(19) United States
(12) Patent Application Publication
(10) Pub. No.: US 2025/0222007 A1
(43) Pub. Date: Jul. 10, 2025

(54) PREPARATION OF LOW MOLECULAR WEIGHT POLYLACTIC ACID AND THE USE THEREOF

(71) Applicant: AL Andalous for Pharmaceutical Industries, Cairo (EG)

(72) Inventors: Ahmed Fathy Hanafy, Alexandria (EG); Mohamed Ahmed Ibrahim, Giza (EG)

(21) Appl. No.: 19/003,145

(22) Filed: Dec. 27, 2024

Related U.S. Application Data
(60) Provisional application No. 63/615,580, filed on Dec. 28, 2023.

(51) Int. Cl. A61K 31/55; A61K 47/10; A61K 47/20; A61K 47/34

(52) U.S. Cl. CPC A61K 31/55; A61K 47/10; A61K 47/20; A61K 47/34

(57) ABSTRACT

Provided herein are methods and compositions related to a pharmaceutical composition for eye injection to certain ocular cavities and/or eye surfaces, or upon injection in ocular tissues. The pharmaceutical composition comprises an active pharmaceutical ingredient and a low molecular weight crystalline biodegradable polymer and a biocompatible solvent.

### RELATED APPLICATIONS

This application claims the benefit of priority to U.S. Patent Application No. 63/615,580, filed Dec. 28, 2023, the content of which is hereby incorporated by reference in its entirety.

### BACKGROUND

Ocular diseases are commonly treated by the instillation of eye medications. These medications usually undergo fast washout and can hardly reach the retina, vitreous body, and rest of the ocular areas due to the greater diffusional distance and anatomical and physiological barriers in the eye. Moreover, even parenteral systemic medications are not able to reach these ocular areas in sufficient concentrations due to the blood-aqueous barrier. This fact represents a serious problem, especially while dealing with chronic retinal diseases which lead to visual impairment and blindness worldwide. Therefore, it requires sustained exposure to the drug at the site of infection. The most prevalent retinal diseases that result in blindness include glaucoma, endophthalmitis, age-related macular degeneration, diabetic retinopathy, retinal vein occlusion, uveitis, infectious retinitis, retinal detachment, and hereditary degenerative diseases including retinitis pigmentosa.

Drug delivery to target tissues in the eye via intravitreal injections of medicines has also been investigated. However, a lot of intravitreally administered medications, notably corticosteroids, and low molecular weight medications, have short half-lives of 2 to 6 hours. In order to maintain therapeutic drug concentrations, frequent injections may be required, which typically increases the risk of serious adverse events like retinal detachment, endophthalmitis, and vitreous hemorrhage as well as negative anterior-segment manifestations like cataract formation and elevated intraocular pressure.

Ocular conditions are diseases that affect the eye, or parts of the eye. According to the region affected, there are two different types of conditions that affect the eye, an anterior (i.e., front of the eye) ocular condition or a posterior ocular condition. An anterior ocular condition is a disease, or a condition that affects an anterior ocular region. The posterior ocular condition is a disease that primarily affects posterior ocular site, such as the choroid or sclera. Endophthalmitis is a disease that affects the posterior ocular region and causes a harmful inflammation in the intraocular fluids and tissues that in most cases may lead to loss of vision and it is considered one of the most serious complications of ophthalmic surgery.

A common problem regarding ocular conditions is the difficulty of delivering the therapeutic dose of the drug to the back of the eye from the plasma because the contact of the medication with the intraocular region is very limited. Therefore, physicians aim to apply the medication directly into the affected area and need to extend the duration of the drug in the vitreous humor after injection to avoid repeated doses and minimize injuries in the eye.

Accordingly, there is a need for a biodegradable implant that would overcome most of the problems faced with the above-discussed implantable dosage forms.

### SUMMARY

The proposed solution includes the development of an in situ forming biodegradable implant comprising at least one pharmaceutically acceptable solvent and a polymer of low molecular weight. The polymer may be commonly known to be highly safe, e.g., a polymer with Generally Recognized as Save status according to the USFDA, or that is used in FDA-approved products. The polymer may have a particular degree of crystallinity to provide better control over the release of a versatile range of medications. The pharmaceutical compositions described herein provide an ease of injection or topical application and tailored solidification to semi-solid or solid implant.

In one aspect, disclosed herein are pharmaceutical compositions, preferably for ocular injection or topical application. The pharmaceutical composition may comprise an active pharmaceutical ingredient, a low molecular weight partially crystalline biodegradable polymer, and a biocompatible solvent. In some embodiments, the pharmaceutical composition is administered for ophthalmic care. In some embodiments, the low molecular weight crystalline biodegradable polymer is poly-L-lactide (PLLA). The pharmaceutical composition may comprise about 20% to 50% w/w PLLA, such as 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%, 50% w/w PLLA.

In some embodiments, the low molecular weight crystalline biodegradable polymer is bioresorbable. In some embodiments, the biocompatible solvent is N-methyl-2-pyrrolidone (NMP), propylene glycol, acetone, dimethyl sulfoxide (DMSO), tetrahydrofuran, 2-pyrrolidone, chloroform, ethyl acetate, polyethylene glycol (PEG), or triacetin. Preferably, the biocompatible solvent is DMSO. In some embodiments, the pharmaceutical composition comprises about 40% to 80% w/w DMSO, such as 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% w/w DMSO.

In some embodiments, the biocompatible solvent is PEG, such as PEG 4000 or PEG 1500. In some embodiments, the pharmaceutical composition comprises about 1% to 20% w/w PEG, such as 1%, 5%, 6%, 7%, 8%, 9%, 10%, 11%, 12%, 13%, 14%, 15%, 16%, 17%, 18%, 19%, or 20% w/w PEG. Preferably, the pharmaceutical composition comprises 10% w/w PEG.

In some embodiments, the active pharmaceutical ingredient is ciprofloxacin, delafloxacin, levofloxacin, ofloxacin, gemifloxacin, moxifloxacin, or besifloxacin. The pharmaceutical composition may be administered for ocular injection or topical application. For example, the pharmaceutical composition may be administered to an ocular cavity, eye surface, or ocular tissue. In some embodiments, the pharmaceutical composition is in the form of an injectable solution, suspension, emulsion or dispersion, and/or the pharmaceutical composition is in the form of an in-situ forming implant composition. In some embodiments, the pharmaceutical composition delivers the active pharmaceutical ingredient in controlled, delayed, and/or sustained delivery. In some embodiments, the pharmaceutical composition treats an ocular disease, such as glaucoma, endophthalmitis, age-related macular degeneration, diabetic retinopathy, retinal vein occlusion, uveitis, infectious retinitis, retinal detachment, or retinitis pigmentosa.

In some embodiments, the crystalline biodegradable polymer has crystallinity between about 10%-50%. In some embodiments, the crystalline biodegradable polymer has crystallinity of 10%, 15%, 20%, 25%, 30%, 35%, 40%, 45%, or 50%.

In one aspect, disclosed herein are methods of treating an ocular disease in a subject in need thereof. The methods may comprise administering to said subject the pharmaceutical composition disclosed herein, optionally via an injection or topical application (e.g., topically in eye contour). The eye contour is the area around the eyes that includes the eye socket, eyelids, and under-eye area. In some embodiments, the ocular disease is glaucoma, endophthalmitis, age-related macular degeneration, diabetic retinopathy, retinal vein occlusion, uveitis, infectious retinitis, retinal detachment, or retinitis pigmentosa. In some embodiments, the injection is administered intravitreally, intramuscularly, intracamerally, periocularly, subchoroidally, subconjunctivally, subretinal, under eye mucosal layer, or utilizing pre-injected watery bleb under the surface of the eye. In some embodiments an injection having a volume of 1 to 50 μl, in particular 2 to 25 μl, is given intravitreally or subconjunctivally. In some embodiments, the injection is given to the eye as needed or regularly between once a week and annually, in particular once each 3rd to 6th month.

In one aspect, disclosed herein are methods of administering to a subject the pharmaceutical composition disclosed herein, optionally via an injection or topical application (e.g., topically in eye contour). The injection may be administered intravitreally, intracamerally, periocularly, subchoroidally, subconjunctivally, intramuscularly, subretinally, under eye mucosal layer or utilizing pre-injected watery bleb under the surface of the eye. In some embodiments, an injection having a volume of 1 to 50 μl, in particular 2 to 25 μl, is given intravitreally or subconjunctivally. In some embodiments, the injection is administered to the eye as needed or regularly, e.g., between once a week and annually, in particular once each 3rd to 6th month.

### DESCRIPTION OF THE FIGURES

FIG. 1 show release of besifloxacin in simulated tears fluid pH 7.2.

FIG. 2 shows DSC thermogram of poly L-lactic acid (PLLA) used in the implant disclosed herein. Tg is around 43.7° C. and the degree of crystallinity is about 45%.

FIG. 3 shows DSC thermogram of one PLLA grade used in the one of implant trials. Tg is around 46.3° C. and the degree of crystallinity is about 15%.

### DETAILED DESCRIPTION OF THE INVENTION

In some aspects, described herein are in situ forming biodegradable ocular implants and uses thereof in treating a medical condition of the eye. The in situ forming implants are designed to be monolithic, i.e., the active agent is dissolved or dispersed in the solution containing the biodegradable polymer matrix and a biocompatible solvent. Furthermore, the implants are formed to release the active agent into an ocular region over a period of time.

The disclosed pharmaceutical composition aims to use biodegradable polymer that may be semi-crystalline in order to achieve more stability and controllable release rates of the active agent. The low molecular weight polymer matrix may be semi-crystalline and compatible with the selected site of implantation to achieve suitable drug loading for treatment of eye conditions with controlled burst effect while offering controlled release of the medication. Semi-crystalline low molecular weight polymers offer superior stability and slower biodegradability of polymer and accordingly decrease any side effects that may arise from acidity caused by degradation of polymer in comparison with amorphous ones. Moreover, the low molecular weight polymer matrix provides suitable syringeability due to low molecular weight viscosity and more control of medication release and lower burst effect due to semicrystalline structure. Preparations may contain poly L-lactic acid (PLLA) in about 10-90% w/w. The polymer concentration maybe between about 30-50% w/w. DMSO can efficiently solubilize the PLLA and produce consistent in situ forming implant body when used in 30%-70% w/w ratio or 40%-60% w/w. PEG of different grades might be used in concentrations of 1% to 20% w/w or 1-10% w/w.

The implant construction typically varies according to the desired drug release profile, the particular active pharmaceutical ingredient (API) used, and the condition being treated. API that may be used include but are not limited to, an antibiotic, a drug that reduces ocular surface inflammation, a drug that reduces elevated intra-ocular pressure (IOP) that can be applied to the eye, an anti-inflammatory agent, an anti-infective agent, an antitumor agent, an antiviral agent, a lubricating agent, an endogenous cytokine, an ace inhibitor, an agent that influences basement membrane, an agent that influences the growth of endothelial cells, an anti-hypertensive agent, an adrenergic agonist or blocker, a cholinergic agonist or blocker, an aldose reductase inhibitor, an anesthetic, an anti-allergic agent, or a steroidal or non-steroidal anti-inflammatory agent. The API may be a quinolone derivative, such as ciprofloxacin, delafloxacin, levofloxacin, ofloxacin, Gemifloxacin, moxifloxacin, or Besifloxacin. The API may be an antibacterial agent, which may constitute about 1%-80% by the weight of the implant. In some embodiments, the API is from 10% to 50% by weight of the implant. In some embodiments, the API is from 2% to 40% by weight of the implant.

### Definitions

For convenience, certain terms employed in the specification, examples, and appended claims are collected here.

As used herein, the singular forms “a”, “an,” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising.” The transitional terms “consist” and any grammatical variations thereof, are intended to be limited to the elements stated in the claims and exclude any elements not stated in the claims. The phrases “consisting essentially of” and any grammatical variant thereof indicate that the claim encompasses embodiments containing the specified elements and includes additional elements that do not materially affect the basic and novel characteristic(s) of the claim.

The term “about” or “approximately” means within an acceptable error range for the particular value as determined by one of ordinary skill in the art, which will depend in part on how the value is measured or determined, i.e., the limitations of the measurement system, and the conventional variability accepted in the art for the concerned parameter.

As used herein, the term “administering” means providing a therapeutic agent or composition to a subject, and includes, but is not limited to, administering by a medical professional and self-administering.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The term “preventing” is art-recognized, and when used in relation to a condition is well understood in the art and includes administration of a composition which reduces the frequency of, or delays the onset of, symptoms of a medical condition in a subject relative to a subject which does not receive the composition. Thus, prevention of loss of vision includes, for example, reducing the incidence, number, and/or size of loss of vision in a population of patients receiving a prophylactic treatment relative to an untreated control population, and/or delaying the loss of vision in a treated population versus an untreated control population, e.g., by a statistically and/or clinically significant amount.

Viscosity: As used herein, viscosity stands for dynamic viscosity measured at 25° C. using a rheometer and extrapolating shear rate to 0 s1.

In certain embodiments, a therapeutic agent may be used alone or conjointly administered with another therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In certain embodiments, conjoint administration of the combinations of compositions of the invention with one or more additional therapeutic agent(s) (e.g., one or more additional chemotherapeutic agent(s)) provides improved efficacy relative to each individual administration of the combinations of compounds of the invention or the one or more additional therapeutic agent(s). In certain such embodiments, the conjoint administration provides an additive effect, wherein an additive effect refers to the sum of each of the effects of individual administration of the combinations of compositions of the invention and the one or more additional therapeutic agent(s).

The term “effective amount” or “therapeutically effective amount,” as used herein, means an amount of a composition of the present invention, that, when administered to a subject (e.g., a human subject or an animal model) in need of such treatment, is sufficient to effect treatment, as defined herein. For example, an effective amount or therapeutically effective amount of the compositions described herein, when administered to a subject (e.g., a human subject or an animal model), is sufficient to treat an ocular disease.

“Subject” refers to an animal, such as a mammal, for example a human. The methods described herein can be useful in both humans and non-human animals. In some embodiments, the subject is a mammal (such as an animal model of disease), and in some embodiments, the subject is human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla or a chimpanzee).

### Pharmaceutical Compositions

The compositions and methods of the present invention may be utilized to treat an individual in need thereof.

The present disclosure relates to the synthesis, formulation, and production of low molecular weight biodegradable polymers with a specified degree of crystallinity that is suitable for sterilization and uses for in situ forming implants. The types of implants covered by this invention includes solids and semisolids that undergo formation by prior treatment, or upon delivery to certain ocular cavities and/or eye surfaces, upon topical application to ocular cavities and/or eye surfaces, or upon injection in ocular tissues to deliver different medications in variable concentrations to localized ocular area(s), while delivering drugs in a sustained and/or controlled release manner ranging from minutes to weeks.

In situ forming implant (ISFI) drug delivery systems provide a means by which a controlled release depot can be physically inserted into a target site without the use of surgery. ISFI avoid the use of large needles or microsurgery, and they are injected as low viscous solutions and transform in the body to a gel or solid depot. Different triggers can be used to stimulate this transformation: (1) in situ cross-linking, (2) in situ solidifying organogels, and (3) in situ phase separation.

Biodegradable polymer-based drug delivery systems hold great promise for the treatment of ocular illnesses, offering a potential solution to many of the limitations of conventional (i.e., systemic, oral, and topical) methods of ophthalmic drug administration.

The present disclosure encompasses preparation of an injectable drug delivery depot ‘in-situ forming biodegradable implant’ for treating a medical eye condition especially a condition that threatens vision and requires long-term therapy. This implant may comprise an active agent dissolved in a solution of a biodegradable polymer matrix with biocompatible solvent forming a viscous syringeable solution. Solvent has solubilizing properties. Once the solution is injected and in contact with eye fluids, the solvent rapidly diffuses from the polymer, which leads to polymer precipitation and a depot forms.

Unlike previous in situ forming implants, the implant disclosed herein is designed to be compatible and suited for ocular administration. The design includes the selection of the active pharmaceutical ingredient (API) itself, the solvent used to solubilize the polymer, the biodegradable polymer, and the catalyst involved in the preparation of the biodegradable polymer. The API may be an ocular medication, and/or approved by the common regulatory agencies. The API may have a specific particle size, and/or may be non-irritant and sufficiently stable. In addition, the composition is selected so that the initial burst release of the API will not exceed its maximum daily dose to avoid dose related toxicity. Moreover, the solvent involved in the formula should be compatible to the eye and may be approved by the common regulatory agencies for ophthalmic administration. The solvent may have a beneficial therapeutic impact that synergizes with the pharmacological activity of the API. Further, the quantity of API released may be monitored during the implantation process (e.g., to determine the amount of the initial burst release). The selection of the polymer may be based in part on compatibility of the polymer, its solubility in the solvent of the formula, molecular weight, degree of crystallinity, syringeability, and the catalyst involved in the synthesis process. The catalyst may have a therapeutic benefit to the eye and approved by US-FDA excipient database for ophthalmic administration. Preferably, the selected biodegradable polymer may be low or free from elemental impurities, residual solvents and able to be sterilized and/or purified to reduce endotoxins below a suitable threshold.

Low molecular weight crystalline biodegradable polymers, such as poly L-lactic acid (PLLA), could be used as the degradable polymer instead of amorphous biodegradable polymers, such as poly DL-lactic acid (PDLLA). The degree of crystallinity greatly affects the kinetic release behavior and the stability of the implant. Generally, high molecular weight polymers have lower release kinetics than low molecular weight ones. On the other hand, lower crystallinity means faster release kinetics even with high molecular weight polymer, and higher crystallinity means slower release kinetics even with low molecular weight polymers. Another aspect that is affected by crystallinity is the stability of the implant. In general, crystalline PLLA has a longer lifetime than amorphous PDLLA. Therefore, a degree of crystallinity may be selected to achieve the desired release-duration target.

The implants disclosed herein can be used to deliver drugs to treat an ocular disease. The ocular disease may be a disease that negatively impacts vision. The ocular disease may be endophthalmitis, macular edema, retinal disorders, diabetic retinopathy, retinal detachment, central retinal vein occlusion, age-related macular degeneration, or uveitis retinal disease. Endophthalmitis is a purulent inflammation of the intraocular fluids (vitreous and aqueous) usually due to bacterial or fungal infections which causes blindness. Acute cases of endophthalmitis are caused by bacterial or fungal infections and the symptoms are often observed within several weeks (commonly 6 weeks) after surgery or trauma to the eye. Endophthalmitis is commonly treated by repeated injections of an antibacterial agent mixed with an antifungal agent mainly by the intravitreal route to control the infection. These repeated doses are harmful to the eye tissues as they may cause retinal detachment. Therefore, the doses are combined in one single injection in the form of the biodegradable in situ forming implant disclosed herein. The implant remains in the eye to deliver the API for a period ranging from few minutes to several weeks in a sustained and/or controlled manner.

The bio-erodible in-situ forming biodegradable implant may form solid and/or semisolid implants upon delivery. This implant may be designed to give variable accessibility to the physician from delivery to certain ocular cavities and/or eye surfaces or implantation—according to the patient situation—in different ocular regions. This implant could be delivered to the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, the macula, or the retina. The implant may be delivered into a suprachoroidal space, where the sclera is cut to expose the suprachoroid. In some embodiments, the suprachoroidal space is a surgically induced avascular region.

An active pharmaceutical ingredient (API) is the main ingredient in a medicine that produces a desired effect. APIs can be found in tablets, capsules, creams, or injectables. APIs can be chemically synthesized or sourced from nature.

The active pharmaceutical ingredient (API) may be selected from the group consisting of antibiotics, drugs that reduce ocular surface inflammation, and/or reduce elevated intra-ocular pressure (IOP) that can be applied to the eye and treat the target disease.

The active pharmaceutical ingredient (API) used in the controlled release pharmaceutical compositions disclosed herein may include one or more anti-VEGFs, VEGFs, Tyrosine kinase inhibitors, antiparasites, H2 receptor antagonists, antimuscarinics, prostaglandins and prostaglandin analogues, non steroidal anti-inflammatory agents, corticosteroids, chelating agents, cardiac glycosides, phosphodiesterase inhibitors, thiazide, anesthetic agents, carbonic anhydrase inhibitors, antihypertensives, anti-cancers, calcium channel blockers, analgesics, opioid antagonists, antiplatelets, anticoagulants, fibrinolytics, statins, adrenoceptor agonists, beta blockers, antihistamines, mucolytics, decongestants, central nervous system agents, 5HT1 antagonists, opiates, 5HT1 agonists, antiepileptics, dopaminergics, antibiotics, antifungals, anthelmintics, antivirals, antiprotozoals, antidiabetics, insulin and its derivatives, GLP-1 receptor agonists, antiestrogens, posterior pituitary hormone antagonists, peptide drugs, protein drugs, protein kinases, antigens, antidiuretic hormone antagonists, bisphosphonates, dopamine receptor stimulants, androgens, steroid reductase inhibitors, non-steroidal anti-inflammatoires, immunosuppressants, local anaesthetic, sedatives, antipsoriatics, silver salts, topical antibacterials, vaccines, or vaccine antigens.

The active pharmaceutical ingredient (API) may include one or more tyrosine kinase inhibitors: 3-[4-(1-formylpiperazin-4-yl)-benzylidenyl]-2-indolinone, Acalabrutinib, Afatinib, Alectinib, Axitinib, Axotinib, Bosutinib, Brigatinib, Cabozantinib, Canertinib, Capmatinib, Cediranib, Ceritinib, Crenolanib, Crizotinib, Dabrafenib, Dacomitinib, Dasatinib, Dovitinib, Entrectinib, Erlotinib, Fedratinib, Flumatinib, Foretinib, Fostamatinib, Gefitinib, Geldanamycin, Genistein, Gilteritinib, Glesatinib, Ibrutinib, Icotinib, Imatinib mesylate, Lapatinib, Larotrectinib, Lestaurtinib, Lorlatinib, Lenvatinib, Midostaurin, Motesanib, Neratinib, Nilotinib, Nintedanib, Osimertinib, Pacritinib, Pazopanib, PD173955, Pexidartinib, Piceatannol, Ponatinib, Radicicol, Radotinib, Regorafenib, Ruxolitinib, Selpercatinib, Saracatinib, Savolitinib, Selumetinib, Sorafenib, Sunitinib, Tandutinib, Tesevatinib, Trametinib, Tucatinib, Vandertanib, Vatalanib, Vemurafenib.

In some embodiments, the active pharmaceutical ingredient (API) includes one or more prostaglandins: Naturally occurring prostaglandins, Alprostadil, Bimatoprost, Carboprost, Cloprostenol, Dinoprostone, Enprostil, Fenprostalene, Fluprostenol, lloprost, Latanoprost, Latanoprostene bunod, Luprostiol, Misoprostol, Netarsudil, Prostalene, Tafluprost, Travoprost, Unoprostone, Other preferred active pharmaceutical ingredients include dexamethasone and cyclosporine. In some embodiments, the active pharmaceutical ingredient includes one or more anti-VEGFs: Adalimumab, Aflibercept, Anecortave, Bevacizumab, Brolucizumab, Etanercept, Infliximab, Pegaptanib, Ranibizumab, Verteporfin and their biosimilars.

In some embodiments, the loading of the active pharmaceutical ingredient (API) varies between 0.1 wt % and 90 wt % of the total weight of the polymer-solvent mixture, preferably 0.2 wt % to 50 wt %, more preferably 0.5 wt % to 30 wt %, most preferably from 1 wt % to 10 wt %. In some embodiments, the loading of the active pharmaceutical ingredient (API) is 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, or 90 wt %.

The implant may comprise a polymer selected from any one of poly DL-lactic acid, poly DL-lactide-co-glycolide, and poly lactide co-e-caprolactone. In some embodiments, the polymer is poly L-lactide-co-glycolide. In some embodiments, the polymer is poly L-lactic acid.

The polymer may be synthesized with precise characteristics to achieve safety and compatibility with the eye. Therefore, organic solvents and elemental impurities may be avoided during the synthesis and purification process. Instead, zinc chloride may be used to be the polymerization catalyst and ring opening initiator as it is not only compatible to the eye and listed in the FDA database of excipient for ophthalmic preparations, but also considered as an important elemental supplement that the eye tissues should contain. The polymer may be dissolved in a water miscible biocompatible solvent. Solvents that are involved in this formula are, but not limited to N-methyl-2-pyrrolidone (NMP), propylene glycol, acetone, dimethyl sulfoxide (DMSO) tetrahydrofuran, 2-pyrrolidone, or triacetin.

The term “biocompatible solvent” takes its usual definition in the art and so refers to a solvent that is not harmful or toxic to living tissue. In some embodiments, the “biocompatible solvent” is miscible with water, in particular it is miscible with water in all proportions. In some embodiments, the “biocompatible solvent” is further capable of dissolving, partially or wholly, the biodegradable polymer. In some embodiments, the biocompatible solvent is a liquid capable of dissolving at least 1 mg/ml of the purified polyester at 35 to 37° C., and capable of dissolving, dispersing or suspending at least one active pharmaceutical ingredient. In some embodiments, the biocompatible solvent is selected from N-methyl-2-pyrrolidone, triacetin, dimethylsulfoxide, anisole, benzyl benzoate, benzyl alcohol, acetone, butyl acetate, propyl acetate, ethyl acetate, methyl acetate, ethyl formate, isopropyl acetate, glycofurol or combinations thereof. In some embodiments, the biocompatible solvent is selected from dimethylsulfoxide, optionally in combination with benzyl benzoate.

The implant may comprise a biodegradable polymer, solvent system, and active agent. In some embodiments, variable cosolvents are added to enhance the solubility of the polymer and the API in the solvent system. In addition, these cosolvents may ameliorate the initial burst release.

In some embodiments, the dynamic viscosity, at 25° C., of an injectable composition is between 10 and 100,000 mPas.s. In some embodiments, the viscosity is between 100 to 3000 mPas.s between 200 to 2000 mPas.s. In some embodiments, the dynamic viscosity, at 25° C., of an injectable composition is about 800 to 1200 mPas.s.

A detailed method for making a bio-erodible implant for treating a medical condition of the eye involve many procedures and details that can be summarized in the following steps of: (a) synthesis of the polymer (b) dissolving the polymer in the solution of the solvent system (c) dissolving with the API in specified solvent system, and (d) sterilization of the formula in selected primary packaging material.

### Methods of Treatment

Provided herein are methods of preventing or treating an ocular disease comprising administering a composition described herein. In some embodiments, the ocular disease is glaucoma, endophthalmitis, age-related macular degeneration, diabetic retinopathy, retinal vein occlusion, uveitis, infectious retinitis, retinal detachment, or retinitis pigmentosa.

In some embodiments, a method of treating a subject in need thereof with a controlled release pharmaceutical composition. The pharmaceutical composition may be administered for eye injection, in any part of the eye, for ophthalmic care, for topical application, or in therapy of ophthalmic conditions. Ophthalmic care is a means to treat the eye by applying medications. The method may comprise administering to said subject an injection or a topical application of the composition as disclosed herein. In some embodiments, the injection is given to the eye intravitreally, intracamerally, periocularly, subchoroidally, subconjunctivally, subretinally, intramuscularly, or under eye mucosal layer or utilizing pre-injected watery bleb under the surface of the eye. Such a bleb will enhance the solidifying of the composition.

In some embodiments, the injection volume varies between 1 microliter and 1000 microliter, preferably between 2 to 500 microliter. For example, the injection volume can be in the range from 3 microliter to 100 microliter, more preferably from 5 microliter to 50 microliter, most preferably from 10 microliter to 30 microliter. For an intravitreally given injections volumes in the range from 5 to 25 μl are preferred, for subjconjunctivally given injections volumes in the range of 2 to 15 μl are preferred. In some embodiments, an injection having a volume of 1 to 50 μl, in particular 2 to 25 μl, is given intravitreally or subconjunctivally. In some embodiments, the injection is given to the eye as needed or regularly between once a week and annually, in particular once each 3rd to 6th month.

In some embodiments, the compositions described herein are particularly useful in reducing intraocular pressure, especially intraocular pressure which is associated with glaucoma. The injectable ophthalmic compositions described herein provide a steady release of an amount of API or combination of APIs which is effective in treating elevated intraocular pressure. The daily dosage and drug release duration is controlled by formulation composition and injection volume.

In some embodiments, the injection is administered intravitreally, intracamerally, periocularly, subchoroidally, subconjunctivally, intramuscularly, subretinally, under eye mucosal layer or utilizing pre-injected watery bleb under the surface of the eye. Intravitreal injection is a procedure to place a medication directly into the space in the back of the eye called the vitreous cavity, which is filled with a viscous fluid called the vitreous humor. Intracameral injection is an injection of a substance into the eye anterior chamber. Topical application is the application of a disclosed composition to the surface of the eye. In situ forming implant (ISFI) drug delivery systems provide a means by which a controlled release depot can be physically inserted into a target site without the use of surgery. ISFI avoid the use of large needles or microsurgery and they are injected as low viscous solutions and transform in the body to a gel or solid depot. Different triggers can be used to stimulate this transformation: (1) in situ cross-linking, (2) in situ solidifying organogels, and (3) in situ phase separation.

### The Biodegradable Polymer System

The polymer implant system consists of a biodegradable polymer combined with an API that is either dissolved or suspended in a biocompatible solvent to create a homogenous or heterogenous polymer implant system. When the formula comes into contact with biological aqueous fluid or is injected into body tissues or organs, solvents escape the system and water may diffuse into the polymer matrix. As a result, the polymer precipitates, entrapping the drug substance and forming a depot implant at the site of administration. This system uses biodegradable polymers to avoid invasive surgical procedures to remove the implant from the site of administration. Suitable bio-degradable polymers include polyhydroxy acids, polyanhydrides, polyorthoesters, polyesteramides, and others. Taking into account the degradation characteristics and approval by the USFDA, Poly lactides, lactide/glycolide copolymers, and lactide/caprolactone copolymers are most commonly used. The rate of degradation can be controlled by varying the polymer type, and polymer concentration or using a combination of polymers in different ratios.

Biodegradable polymers may be used as resorbable sutures, for prosthetics purposes especially in tissue regeneration scaffold implants, for drug delivery purposes and/or a combination thereof. These biodegradable polymers were mainly synthetic aliphatic-aromatic polyesters such as Polylactide, Polyglycolide, Poly-ε-caprolactone, polyhydroxy alkanoates and copolymers and blends of variable configuration and composition. Typical examples for polyester blends for drug delivery were described in patent WO9615173A1, and U.S. Pat. No. 5,883,199.

Biocompatible polymer: Polymer that does not produce toxic or harmful products and stimulate an immune response in biological systems.

Biodegradable & Bioresorbable polymer and polyester: As used herein, the term “bioresorbable polymer” refers to a polymer that is eroded by cellular activity and/or dissolution in a biological system such as the human body or in vivo. Typically, the present bioresorbable polyesters degrade in vivo by hydrolysis of their ester backbone into non-toxic products.

In some embodiments, PLA is the polymer in the pharmaceutical composition described herein. In general, Polylactic acid is a widely used biodegradable polymer that is approved by the FDA and may be used as an ocular in-situ forming implant. Various characteristics and parameters of the PLA can be chosen to improve the characteristics of the implants discussed herein, for example, the molecular weight, crystallinity, the synthesis process technique, the catalyst used in the synthesis process, the solvent used to dissolve the polymer, and the ratio of drug loaded into the PLA. In addition, polymer characteristics that are considered include but are not limited to, biocompatibility and biodegradability at the site of implantation, compatibility with the API, and processing temperature.

The following bioresorbable polymers, copolymers and terpolymers may be used for controlled drug delivery composition: Polylactide (i.e., poly(lactic acid), PLA), polyglycolide (PGA) and poly(e-caprolactone) (POL), polydioxanone (PDO), polytrimethylenecarbonate (PTC), polylactides (PLA), poly-L-lactide (PLLA), poly-DL-lactide (PDLLA), PolyL-DL-lactide (PLDL), stereocopolymers; copolymers of glycolide, glycolide/trimethylene carbonate copolymers (PGA/TMC); other copolymers of PLA, such as lactide/tetramethylglycolide copolymers, lactide/trimethylene carbonate copolymers, lactide/d-valerolactone copolymers, lactide/e-caprolactone copolymers, L-lactide/DL-lactide copolymers, glycolide/L-lactide copolymers (PGA/PLLA), polylactide-co-glycolide; terpolymers of PLA, such as lactide/glycolide/trimethylene carbonate terpolymers, lactide/glycolide/e-caprolactone terpolymers, PLA/polyethylene oxide copolymers; polydepsipeptides; unsymmetrically (3,6-substituted)-poly-1,4-dioxane-2,5-diones; polyhydroxy-alkanoates, such as polyhydroxybutyrates (PHB); PHB/b-hydroxyvalerate copolymers (PHB/PHV); poly-b-hydroxypropionate (PHPA); poly-p-dioxanone (PDS); poly-d-valerolactone-poly-e-caprolactone, poly(e-caprolactone-DL-lactide) copolymers; methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PVA); polypeptides; poly-b-malic acid (PMLA); poly-b-alkanoic acids; polycarbonates; polyorthoesters; polyphosphates; poly(ester anhydrides); and mixtures thereof; and natural polymers, such as sugars, starch, cellulose and cellulose derivatives, polysaccharides, collagen, chitosan, fibrin, hyaluronic acid, polypeptides and proteins.

The number average molecular weight of the polyester typically varies in the range of 1,000 g/mol to 250,000 g/mol, depending on the configuration and shape of the polyester molecule. In some embodiments, the molecular weight of polylactides or lactide and glycolide copolymers is about 1,000 to 250,000 g/mol. In some embodiments, the low molecular weight polylactides or lactide and glycolide copolymers have a molecular weight of about 1,000 to 50,000 g/mol. In some embodiments, the low molecular weight polylactides or lactide and glycolide copolymers have a molecular weight of about 1,000 to 10,000 g/mol, 10,000 to 20,000 g/mol, 20,000 to 30,000 g/mol, 30,000 to 40,000 g/mol, or 40,000 to 50,000 g/mol. In some embodiments, the low molecular weight polylactides or lactide and glycolide copolymers have a molecular weight of about 1,000 g/mol, 5,000 g/mol, 10,000 g/mol, 15,000 g/mol, 20,000 g/mol, 25,000 g/mol, 30,000 g/mol, 35,000 g/mol, 40,000 g/mol, 45,000 g/mol, or 50,000 g/mol. In some embodiments, the polylactides or lactide and glycolide copolymers have a polydispersity index of 1.5 to 2.7, preferably 1.7 to 2.6, preferably 1.9 to 2.5, preferably 2.1 to 2.4, as determined by GPC analysis.

### The Synthesis Process of the Polymer

The polymer used in the compositions described herein may be synthesized by polymer condensation followed by ROP, ring-opening polymerization technique. This technique requires mild conditions and low concentration of the catalyst; however, it typically provides high purity, less side product, and less racemization as well. The first step is the condensation of lactic acid between about 90-200° C. for a duration between about 3-15 h to produce lactides. And more usually between about 120-180° C. the temperature of condensation may be between about 130-160° C. the condensation temperature of the polymer is usually more than about 80° C. and less than 200° C. The purity of lactide may be enhanced by, and not limited to, using a multistage melt crystallizer to remove the traces of lactic acid and oligomers. Then, the initiator, the ring opener, may be added to begin the polymerization of lactides and produce polylactic acid. The initiator is preferably selected not only to be safe and compatible with the eye but also to make it beneficial to the eye tissues as well. Many studies found that tin or Zn produces fewer impurities and higher molecular weight. These catalysts are favored due to their covalent metal-oxygen bond and free p and d orbitals. Tin is an irritant to the eye and not approved by the FDA for ophthalmic preparations, for that reason, it is more convenient to use zinc. zinc catalysts include, but are not limited to, zinc metal, zinc amido-oxazolinate, zinc lactate, zinc alkoxides, [LZn(μ-OBn)]₂, zinc acetate Zn(OAc)₂·2H₂O, β-Pyridylenolate zinc, aza (oxazoline) ligand-based zinc, and Mononuclear zinc β-diketiminates. more usually, ZnCl₂ may be the catalyst because, in a very low concentration, it is particularly desirable in ophthalmic pharmaceutical compositions containing therapeutically active agents. Indeed, it is approved in the FDA excipient database to be used for ophthalmic administration and also provides a source of Zn ion in a concentration that is essential for the healthy eye.

The concentration of the initiator is varied to be of any concentration suitable for eye contact. In some embodiments, the concentration of the catalyst may be between about 1-30% w/w, and more usually between about 1-10% w/w, the concentration maybe between 0.05-5% w/w, and more usually between about ≤1-2% w/w. The concentration of the catalyst may be between about 0.01-1% w/w by adding the catalyst.

### Effect of Temperature

Temperature may be controlled during the entire synthesis process and during cooling. During the condensation step, evaporation of the water, the temperature may be more than 90° C. and may be less than 250° C. These variations may be confirmed by a variety of trials with different temperatures. In some embodiments, the temperature may be higher than 140° C. and the polymer may get slightly yellow to orange before adding the catalyst. In some embodiments, the temperature may be less than 150° C.; in such embodiments, the polymer may show some degree of crystallinity according to thermal analysis using differential scanning colorimeter (DSC) results. In the second step of synthesis and after adding the catalyst, the temperature may be raised to be between about 100-200° C. for the duration between about 3-24 h. In some embodiments, the temperature may be about 200° C. or higher for 7 h to 5 days; in some embodiments, this leads the polymer to become darker and produce a dark red solution. Thermal analysis using DSC showed amorphous and crystalline structure and the sharp peak of crystalline PLLA may disappear. In some embodiments, trials between about 160-190° C. form a yellow solution of PLLA of certain percentage crystallinity that may vary from 5 to 60% calculated using thermal analysis. In some embodiments, temperature is 200° C. or higher.

### Effect of the Catalyst Concentration

Broadly speaking, ophthalmic preparations should have a controlled concentration of elemental impurities in order to be accepted. Therefore, the concentration may be of any concentration compatible with the selected site of implantation, and more usually the concentration is the lowest concentration possible of the catalyst that would be able to achieve the targeted degree of polymerization. Different concentrations may be tried and the results compared according to the differences in molecular weights. In some embodiments, the catalyst maybe zinc metal or zinc lactate and the concentration may be between about 0.01-1% w/w. In some embodiments, the concentration may be between 0.05-5% w/w. In some embodiments, the catalyst may be zinc chloride (ZnCl₂) and the concentration may be between about 0.01%-10% w/w and the molecular weight yielded may be between about 2800-11855 g/mol. And more generally the catalyst may be zinc amido-oxazolinate, zinc alkoxides, [LZn(μ-OBn)]₂, zinc acetate Zn(OAc)₂·2H₂O, β-Pyridylenolate zinc, aza (oxazoline) ligand-based zinc, and Mononuclear zinc β-diketiminates and the concentration may be between about 0.1-5% w/w. In some embodiments, the concentration may be less than 1%, more typically between 0.01%-0.05% w/w. In certain such embodiments, the polymerization yields molecular weight of about 7255-11572.5 g/mol.

### Effect of Time on the Degree of Polymerization

When all other parameters are controlled, then the degree of polymerization may be related to the time of processing, the more time involved in the synthesis process the more molecular weight may be obtained. In some embodiments, the reaction remains for about 5 h after adding the catalyst, and the molecular weight may be between about 2500-3902.5 g/mol. In some embodiments, the reaction time may be 7 h after the catalyst is added and the molecular weight may be between about 7322-8043.7 g/mol. In some embodiments, the process spent may be from 9-10 h and the resulting molecular weight may be about 10544-11572.53 g/mol.

In some embodiments, low molecular weight PLLA may be used as the matrix of the implant with a specified degree of crystallinity that is suitable for sterilization and use for in situ forming implants. Control of the molecular weight may be achieved by varying the concentration of the catalyst, temperature, and time of the polymerization process. The viscosity of the polymer may be determined in a solution of 0.1 g of the polymer in 100 ml chloroform using Ubbelohde capillary viscometer. In combination with the Mark-Houwink equation (M=^{a}\sqrt{[η]/K}), the molecular weights may be determined as a function of processing conditions. Larger duration of the polymerization process may lead to higher molecular weight until a certain point. However, elevated temperatures more than the limit of the desired polymer characteristics may lead to amorphous or semi-crystalline polymorphs of the polymer. The polymer used herein may be low, medium, or high molecular weight with a specific degree of crystallinity. In some embodiments, the polymer may be amorphous. More generally the polymer may be semi-crystalline in order to achieve more stability and controllable release rates of the active agent. In some embodiments, the low molecular polymer matrix may be semi-crystalline and compatible with the selected site of implantation.

### Solvents Used in Ophthalmic Preparations

The solvent involved in this process may possess certain characteristics. It may be nontoxic, nonirritant, approved by the FDA for ophthalmic preparations, and/or water-miscible. Once the polymer solution system contacts with body fluids, which contain a sufficient amount of water, the solvent diffuses away from the polymer to the water and the polymer solidifies to form a solid or semi-solid implant. Solvents that have been used in this approach include N-methyl-2-pyrrolidone (NMP), propylene glycol, acetone, dimethyl sulfoxide (DMSO), tetrahydrofuran, 2-pyrrolidone, chloroform, ethyl acetate, PEG and triacetin. In some embodiments, the solvent is NMP, PEG, or DMSO.

In some embodiments, the solvent system may be PEG and/or DMSO as they have no known risk of keratitis or sensitivity to the eye even in long-term therapy. Experiments show that 0.2 ml/day of 100% DMSO can be administered in the rabbit eye, equal to 4 drops/day for 6 months without any evidence of an adverse effect. In blind human eyes, 66% DMSO (with Tetryzoline) may be applied topically 4-8 times a day. None of any blind eyes evinced any evidence of sensitivity to DMSO, epithelial damage, or an increase of IOP or keratitis, and only one case had a burning sensation. No toxicity was observed for intravitreal injections of miconazole dissolved in 0.1% DMSO in various concentrations (20.5, 5, 10, 30, 40 micrograms). In the lower conjunctival sac, 50 microliters may be instilled directly without any signs of eye irritation or conjunctival or corneal injury. In addition, DMSO and PEG400 are considered water-miscible organic liquids. Therefore, they are dispersible with body fluids. Indeed, these solvent systems may efficiently solubilize the polymer in a ratio up to 10-90% w/w of the entire formula. More usually the solvent system may be between about 10%-70% w/w ratio, and may be between 40%-60% w/w. And more preferably the amount of the solvent system may be less than about 60%. In some embodiments, the ratio of the solvent to the polymer may be less than 50%. The ratio may be between 30-40%.

### Release Rate Study

The implants described herein may be formulated with particles of an active agent dissolved or dispersed in the polymer-solvent solution to make a homogenous or heterogenous implant system. The release of the active agent is achieved by the exchange of solvents that allow the diffusion of the active agent into an ocular fluid. The higher affinity between the solvent and the aqueous phase, i.e., water-miscible solvents, the higher the rate of the phase inversion. Water miscible solvents yield a low viscous solution which allows easy administration of injection and increases biocompatibility by providing a more hydrophilic environment. In general, an undesirable burst effect is associated with the use of such solvents which can reach 70% by the first hour of implantation. The implant system can be modified to control the burst release. In some embodiments, desired burst release is obtained and represents the desirable loading dose of the API by the first day of release. The burst release may be between about 0-90% by the first 24 h of implantation. In some embodiments, the percentage of the initial release maybe between about 40-70% by the first 24 h of implantation. In some embodiments, the initial burst release may be between about 10-40% by the first 24 h of implantation.

The initial burst release of the active agent may depend at least on the time of hardening of the implant and the duration of the solvent phase inversion. More usually the concentration of the solvent system may affect the initial burst release. The initial burst release can be controlled according to the time of hardening of the injected implant, the solubility of the drug, drug loading and their stability. Based on the solubility of the drug in the solvents or solvent mixture or polymer solution, the drug can either be dissolved or dispersed in the formulation. In addition, it can either form a homogenous or heterogenous solution. The release of the API may be faster when the active agent is dispersed in the system rather than dissolved in a polymeric solution. The solubility profile of the APIs in the solvent system is studied and the designed formula is in a ratio of API to the solvent that may be dissolved or suspended. More usually the concentration would be below the saturation capacity of the solvent.

The release kinetics may depend in part on the surface area of the implant. The larger the surface area exposed to the ocular or body fluids, the more the polymer matrix may be eroded, causing faster dissolution of the active agent particles in the fluid. The shape of the implant and the area exposed to the body fluid (i.e., Vitreous humor) may also be varied to control the release rate and the period of treatment. At equal drug loading, larger implants will release a larger dose, but according to the surface to the mass ratio, may deliver a slower release rate.

The release profile can be evaluated in vitro or in vivo by developing several formulae with different active agent concentrations, biodegradable polymer matrixes, and different component ratios.

A USP-approved method for dissolution or release testing (USP 24; NF 19 (2000), pp. 1941-1951) can be used to determine the rate of release in vitro. For instance, a weighted sample of the implant is suspended in simulated tear fluid (STF) at specific pH 7.2. In some embodiments, using the vertical shaking water bath at 37° C., about 250 mg of the implant containing about 2% w/w of the API is injected by a needless syringe in a vial containing 100 ml of STF at pH 7.2. With this amount of solution, the sink condition will be obeyed and the active drug concentration after release will be less than 20% of saturation. The liquid is slowly agitated or shaken in a continuous movement to keep the implants suspended. the release of the dissolved active agent as a function of time can be monitored using various techniques known in the art, such as spectrophotometry, HPLC, mass spectroscopy, and so forth until the solution concentration becomes constant or more than 80% of the active agent has been released.

### Crystallinity

Numerous aspects of the polymer may be influenced by the degree of crystallinity, shape, and thermal properties (e.g., physical, mechanical properties and degradation rates). It has been observed that the degree of crystallinity has a significant impact on the rate of hydrolytic or enzymatic degradability of polylactides in a clinical setting.

Since PLLA has a lower crystallization rate than the majority of semi-crystalline thermoplastics, it is possible to create PLLA samples with a large range of crystallinity, which has significant practical variations. In some embodiments, the polymer may be amorphous. In some embodiments, the polymer is partially crystalline. The release of the active agent from partially crystalline PLLA may be slower than the amorphous structure. Indeed, it may be more stable against enzymatic degradation. In certain embodiments, the glass transition temperature of the partially crystalline form of PLLA is higher than body temperature which leads to stable implant and suitable for sustaining action. In other embodiments, the amorphous form has a glass transition temperature near to the body temperature. In such embodiments, the crystalline form is much more convenient to be implanted in the body.

In cases where the mechanical integrity needs to be sustained, e.g., sutures, orthopedic or dental applications, partially crystalline compositions are preferred. Therefore, controlling the amorphous-crystalline morphology balance is important. It was shown that in the case of PLA this control can be achieved by selecting an appropriate quenching rate, according to the molecular weight of the polymers. The melting enthalpy of a 100% crystalline PLLA may be important for the determination of the crystalline content from the experimental data, while the reported literature values vary in the range between 82-95 J/g.

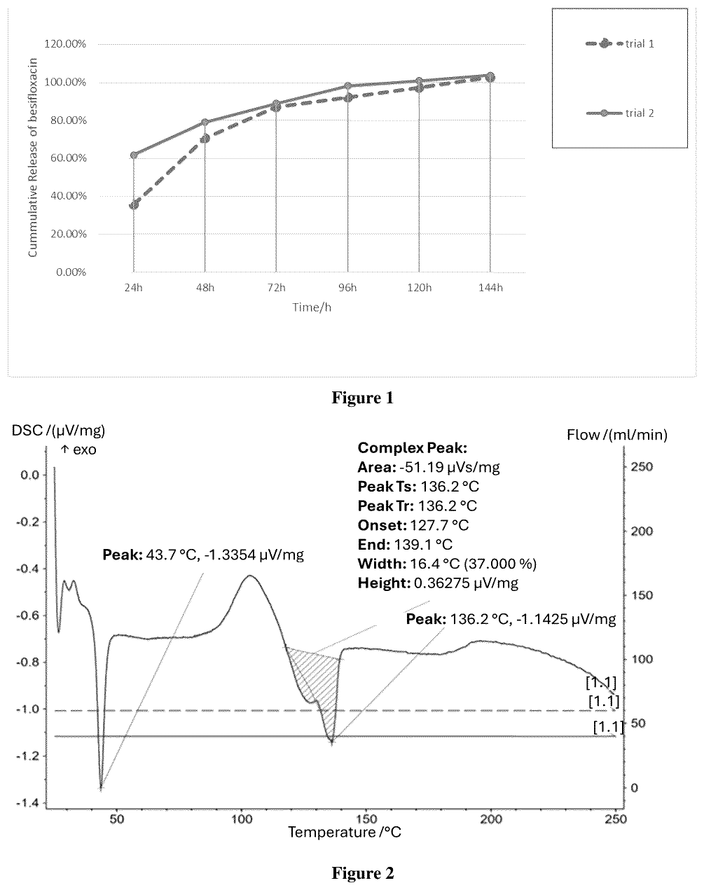

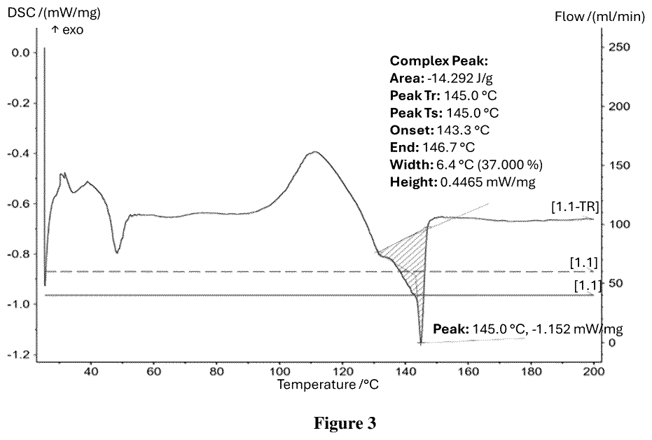

The polymer may be synthesized in a way that controls all factors that may affect the molecular weight of the polymer and also its degree of crystallinity. These factors include the technique of polymerization, the selection of the catalyst, the temperature of the synthesis process, and the time of polymerization. All of these factors come together to produce a preferable low molecular weight polymer with convenient partial crystallinity. In some embodiments, the crystallinity is between about 5%-10%. And more usually the crystallinity is between about 20%-40%. In some embodiments, PLLA is a partially crystalline polymer with a degree of crystallinity between about 10-50%. As shown in the DSC FIGS. 2 and 3 the degree of polymerization is between about 15-50%. As a result, the glass transition temperature is higher than body temperature, between about 43.7-47° C., which leads to stable implant suitable for sustained release. Therefore, these polymers resulted in acceptable release rates shown in FIG. 1.

### Compatibility and Dose Considerations

Pharmaceutical preparations intended to be administered in the eye may be compatible with the eye and/or approved by the FDA for ophthalmic preparation. Compatibility studies should consider the API itself, its particle size, daily dose exposure, metabolism and molecular weight. In some embodiments, besifloxacin is the active agent as it is approved by the FDA for ophthalmic administration, with moderate potency to avoid overdose toxicity and low molecular weight compound. Indeed, in some embodiments, micronized size of besifloxacin is used in order to be less irritant and with good solubility in DMSO/PLLA solution. A suitable daily dose of besifloxacin is typically about 0.9 mg divided in three times per day. In some embodiments, the pharmaceutical preparation contains 5 mg of besifloxacin which is designed to be sustained in the release of the active agent through five days (which is the time typically required to relieve the bacterial infection). Other parameters related to the API include, residual solvent and elemental impurities are considered and limited by USP general chapter <467> and <232> respectively as well as ICH guidelines Q3C and Q3D respectively.

### EXAMPLES

The invention now being generally described, it will be more readily understood by reference to the following examples, which are included merely for purposes of illustration of certain aspects and embodiments of the present invention and are not intended to limit the invention.

### Example 1: Trials

All trials are summarized in table 1.

TABLE 1
Formulations for selected trials studied.

| | Trial 1 | Trial 2 | Trial 3 | Trial 4 |
|---|---|---|---|---|
| | (gm %) | (gm %) | (gm %) | (gm %) |
| Besifloxacin | 2% | 2% | 2% | 2% |
| (BSF) | | | | |
| Dimethyl | 58% | 68% | 50% | 50% |
| sulfoxide | | | | |
| (DMSO) | | | | |
| Poly L- | 40% | 30% | 38% | 38% |
| lactic acid | | | | |
| (PLLA) | | | | |
| PEG 4000 | — | — | 10% | — |
| PEG 1500 | — | — | — | 10% |

### Trial 1

Prepare 58% w/w DMSO in a glass vial or stainless steel container with magnet stirrer or propeller or suitable mixer. Add 2% BSF to DMSO to form a clear pale-yellow solution after approximately 15 min sonication/mixing at temperature 35-40° C. Mill about 40% w/w PLLA till obtaining powder quite coarse or finer, then add it portion by portion to the solution with stirring at temperature 35-40° C. for 30 min, then in sonication/mixing for approximately 60 min. The final appearance is viscous clear solution. This formula showed a preferable initial release. The percentage in vitro cumulative release by the first 24 h may be between about 30-40% and cumulative release was almost complete after 5 to 6 days as shown in FIG. 1. In addition, it has a relatively good injectability and stability profile.

### Trial 2

Prepare 68% w/w DMSO in a glass vial or stainless-steel container with magnet stirrer or propeller or suitable mixer. Add 2% BSF to DMSO to form a clear pale-yellow solution after 15 min sonication at temperature 35-40° C. Mill 30% of PLLA till makes a coarse powder or finer, then add it portion by portion to the solution with stirring at temperature 35-40° C. for 30 min, then in sonication or mixer for about 30 min. The final appearance is viscus clear solution. This formula has a good injectability and low viscosity as it contains greater amount of DMSO. As a result, it showed a high initial burst release as it can reach about 60-70% by the first 24 h and almost complete cumulative API release was reached after 5 to 6 days as shown in FIG. 1.

### Trial 3

Prepare 50% w/w DMSO in a glass vial or stainless steel with a magnet stirrer or mixer. Then add 2% w/w BSF to DMSO to form a clear pale-yellow solution after 15 min sonication or mixing at temperature 35-40° C. Mill 38% w/w PLLA till make a coarse powder or finer, then dry mixing of 10% w/w PEG 4000 and PLLA and add them portion by portion to the solution with stirring at temperature 35-40° C. for about 30 min, then in sonication or mixing for 30 min. The final appearance is a viscous white solution. Although this formula has a relatively high viscosity and showed bad injectability, it showed a high initial first release up to about 50% by the first 24 h and it reached more than about 80% by 48 h of cumulative release.

### Trial 4

Prepare 50% w/w DMSO in a vial with a magnet stirrer or mixer. Add 2% w/w BSF and 10% w/w PEG 1500 to DMSO to form a clear pale-yellow solution after about 15 min sonication or mixing at a temperature 35-40° C. In a mortar, mill 38% w/w of PLLA till make a coarse powder or finer, then add them portion by portion to the solution with stirring at temperature 35-40° C. for about 30 min, then in sonication or mixing for approximately 30 min. Final appearance is viscous white dispersion. The formula showed relatively moderate viscosity and injectability. Nevertheless, it showed a high initial first release up to about 60% by the first 24 h and it reached more than about 80% by 48 h of cumulative release.

### Storage Conditions and Formulations Stability

It is important that the vial should be tightly sealed to avoid evaporation of solvent system from the solution and the formula must be protected from light to avoid degradation of the polymer or the API. Vials were checked by microscope to check for complete solubility if required to be completely soluble and particle size of components if dispersed. Viscosity was also measured for the freshly prepared formula to act as a reference for stability to check for any degradation. Refrigeration at 2-8° C. solidifies the formula and takes about 30 min at hand temperature to liquify. This condition decreases the solubility and separate the API particles from the formula as many particles are appeared in the microscopical analysis. It needs 30-40 mins stirring at 45° C. to restore solubility, and appear nearly clear under the microscope. In some embodiments, storage at 25° C. leads to almost no change in the appearance of the formula (clear light brown viscus solution) and the microscopical analysis shows clear solution without any particulate matters without shaking. More generally, both storage conditions showed no significant changes neither at the physical nor chemical quality of the implant solution for one year, stability for longer time intervals is being studied.

### Polymer Composition

Our synthesized PLLA is a sterile, odorless, white to yellow coarse powder, insoluble in water or alcohol, and soluble in dimethyl sulfoxide and chloroform with pH 3.0-8.6. It must be crystalline from 20-40%, free from residual solvent involved in the synthesis process. Residual monomer NMT 0.5%. No intestinally added catalysts during the manufacturing process except zinc catalyst and its content NMT 0.2% w/w. Bacterial Endotoxin content NMT 13 EU/g of Poly L-lactic acid. the molecular weight of the polymer is from 10000-15000 g/mol. Microbiological attributes is controlled as follow, Total aerobic microbial count: NMT 10³ CFU/g. total yeasts/molds count: NMT 102 CFU/g. Pathogenic organisms: Staphylococcus aureus: absent/g. Escherichia coli: Absent/g. Salmonella: Absent/10 g. Pseudomonas aeruginosa: Absent. Storage Conditions: Store in cool place. Keep the container tightly closed in a dry and well-ventilated place. Handle and store under inert gas. Moisture sensitive. (8-15° C.). Moisture content should be controlled as it affect the stability and shelf life of the polymer. Therefore, water content should be NMT 0.5%.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A pharmaceutical composition comprising an active pharmaceutical ingredient, a low molecular weight crystalline biodegradable polymer, and a biocompatible solvent.

2. The pharmaceutical composition of claim 1, wherein the pharmaceutical composition is administered for ophthalmic care.

3. The pharmaceutical composition of claim 1, wherein the low molecular weight crystalline biodegradable polymer is poly-L-lactide (PLLA).

4. The pharmaceutical composition of claim 3, wherein the pharmaceutical composition comprises about 20% to 50% w/w PLLA.

5. The pharmaceutical composition of claim 4, wherein the pharmaceutical composition comprises 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49%, 50% w/w PLLA.

6. The pharmaceutical composition of claim 1, wherein the low molecular weight crystalline biodegradable polymer is bioresorbable.

7. The pharmaceutical composition of claim 1, wherein the biocompatible solvent is N-methyl-2-pyrrolidone (NMP), propylene glycol, acetone, dimethyl sulfoxide (DMSO), tetrahydrofuran, 2-pyrrolidone, chloroform, ethyl acetate, polyethylene glycol (PEG), or triacetin.

8. The pharmaceutical composition of claim 7, wherein the biocompatible solvent is DMSO.

9. The pharmaceutical composition of claim 8, wherein the pharmaceutical composition comprises about 40% to 80% w/w DMSO.

10. The pharmaceutical composition of claim 9, wherein the pharmaceutical composition comprises 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% w/w DMSO.

11. The pharmaceutical composition of claim 7, wherein the biocompatible solvent is PEG.

12. The pharmaceutical composition of claim 11, wherein the PEG is PEG 4000 or PEG 1500.

13. The pharmaceutical composition of claim 11, wherein the pharmaceutical composition comprises about 1% to 20% w/w PEG.

14. The pharmaceutical composition of claim 13, wherein the pharmaceutical composition comprises 1%, 5%, 6%, 7%, 8%, 9%, 10%, 11%, 12%, 13%, 14%, 15%, 16%, 17%, 18%, 19%, or 20% w/w PEG.

15. The pharmaceutical composition of claim 13, wherein the pharmaceutical composition comprises 10% w/w PEG.

16. The pharmaceutical composition of claim 1, wherein the active pharmaceutical ingredient is ciprofloxacin, delafloxacin, levofloxacin, ofloxacin, gemifloxacin, moxifloxacin, or besifloxacin.

17. The pharmaceutical composition of claim 1, wherein the pharmaceutical composition is administered by ocular injection or topical application.

18. The pharmaceutical composition of claim 1, wherein the pharmaceutical composition is administered to an ocular cavity, eye surface, or ocular tissue.

19-25. (canceled)

26. A method of treating an ocular disease in a subject in need thereof, comprising administering to said subject the pharmaceutical composition according to claim 1.

27-31. (canceled)

32. A method of administering to a subject the pharmaceutical composition according to claim 1.

33-36. (canceled)
